(19) United States
(12) Patent Application Publication
(10) Pub. No.: US 2019/0133472 A1
(43) Pub. Date: May 9, 2019

(54) SYSTEM AND METHOD FOR MONITORING BILIARY OBSTRUCTION

(71) Applicant: AJOU UNIVERSITY INDUSTRY-ACADEMIC COOPERATION FOUNDATION, Suwon-si (KR)

(72) Inventors: Jin Hong KIM, Yongin-si (KR); Myung Hoon SUNWOO, Seoul (KR)

(73) Assignee: AJOU UNIVERSITY INDUSTRY-ACADEMIC COOPERATION FOUNDATION, Suwon-si (KR)

(21) Appl. No.: 16/183,321

(22) Filed: Nov. 7, 2018

(30) Foreign Application Priority Data
Nov. 8, 2017 (KR) 10-2017-0148049

(51) Int. Cl. A61B 5/03; A61B 5/00

(52) U.S. Cl. CPC A61B 5/036; A61B 5/6862; A61B 2560/0219; A61B 5/4222; A61B 5/002

(57) ABSTRACT

According to another exemplary embodiment of the present disclosure, a method for monitoring a pressure in a biliary tract includes: receiving a measured value of a pressure in a biliary tract from a stent for a biliary tract having a pressure sensor mounted therein, by a subcutaneous implant medical device; collecting the measured value of the pressure at a predetermined cycle and transmitting collected information to an external device, by the subcutaneous implant medical device; and analyzing the collected information to expect a timing of biliary obstruction, and providing expected information to the patient, by the external device.

### CROSS-REFERENCE TO RELATED APPLICATIONS

This application claims the priority of Korean Patent Application No. 10-2017-0148049 filed on Nov. 8, 2017, in the Korean Intellectual Property Office, the disclosure of which is incorporated herein by reference.

### BACKGROUND

### Field

The present disclosure relates to a system and a method for monitoring biliary obstruction, and more particularly, to a stent for a biliary tract with a pressure sensor attached thereto by mounting a pressure sensor in a stent for a biliary tract inserted to prevent the biliary obstruction to prevent re-obstruction of the biliary tract and a biliary obstruction monitoring method using the same.

### Description of the Related Art

An average daily bile secretion of an adult is approximately 600 mL to 1 L. However, when a biliary tract (or bile duct) which is a passage of bile is obstructed so that the bile is not smoothly discharged into the intestine, the pressure in the biliary tract is increased, which may cause a severe complication such as jaundice, serious liver damage, cholangitis or sepsis.

As described above, for a conservative treatment of a disease of biliary obstruction, a stent for a biliary tract is widely used to expand the narrowed biliary tract. The stent is a medical device used to be inserted into a narrowed region of the biliary tract to normalize the flow of bile when the biliary tract is obstructed due to postoperative and malignant biliary stenosis and the flow of bile is hindered.

However, as the time elapses, the stent inserted into the biliary tract is occluded due to a bile sludge or tumor overgrowth so that the biliary obstruction may recur. When the biliary obstruction recurs, retreatment for the stent for a biliary tract may be necessary. Further, various complications due to the biliary obstruction may be caused until the retreatment.

However, until now, when the existing stent is used, it is difficult to identify an occluded state of the stent in real time, so that the patient needs to regularly visit the hospital to check the stent. However, in the middle of a visiting cycle of the hospital, there were many cases that severe complications such as jaundice, cholangitis, or sepsis are caused due to the stent occlusion.

When the biliary tract is obstructed in the middle of the visiting cycle of the hospital, the patient visits the hospital after the patient's condition is seriously deteriorated to start the treatment. Therefore, there are problems in that the life of the patient becomes dangerous, a hospitalization period is increased, and a medical expense is increased.

Therefore, studies on a method for simply self-diagnosing whether the biliary tract into which the stent for a biliary tract is inserted is re-obstructed without visiting the hospital are necessary.

### SUMMARY

An object to be achieved by the present disclosure is to provide a system and a method for monitoring biliary obstruction.

Technical objects of the present disclosure are not limited to the aforementioned technical objects and other technical objects which are not mentioned will be apparently appreciated by those skilled in the art from the following description.

According to an aspect of the present disclosure, a system for monitoring a pressure in a biliary tract includes: a stent for a biliary tract including a pressure sensor; and a subcutaneous implant medical device including a communication module which receives a measured value of a pressure in a biliary tract through communication with the pressure sensor and a power module which supplies a power to the pressure sensor.

Desirably, the communication module may communicate with the pressure sensor using anyone of near field communication (NFC) and Bluetooth.

Desirably, the pressure sensor may include a coil which generates an induced current and the power module may wirelessly supply the power to the coil through electromagnetic induction.

Desirably, the power module includes a thin film for harvesting solar energy and supplies the solar energy to the pressure sensor.

Desirably, the subcutaneous implant medical device may further include a storage module and the measured value of the pressure and statistic information of the measured value of the pressure may be stored in the storage module.

Desirably, the subcutaneous implant medical device may be inserted under a subcutaneous tissue of a lateral abdominal region.

Desirably, the system for monitoring a pressure in a biliary tract may further include: an external device which receives the measured value of the pressure from the communication module, analyzes the received measured value of the pressure to expect a timing of biliary obstruction, and provides expected information to a patient.

According to another exemplary embodiment of the present disclosure, a method for monitoring a pressure in a biliary tract includes: receiving a measured value of a pressure in a biliary tract from a stent for a biliary tract having a pressure sensor mounted therein, by a subcutaneous implant medical device; collecting the measured value of the pressure at a predetermined cycle and transmitting collected information to an external device, by the subcutaneous implant medical device; and analyzing the collected information to expect a timing of biliary obstruction, and providing expected information to the patient, by the external device.

Effects according to the present disclosure are as follows:

When the stent for a biliary tract with a pressure sensor attached thereto suggested by the present disclosure is used, a timing when the biliary tract is re-obstructed may be identified in real time. By doing this, the occurrence of severe complications such as liver damage, cholangitis, or sepsis due to the biliary obstruction may be prevented in advance.

Further, the deterioration of the patient's condition due to the biliary re-obstruction is prevented to extend the duration of survival of the patient and reduce the extended hospitalization period and the medical expense for treatment of the complication. Ultimately, it may help the patient to maintain a healthy condition.

Further, when the stent for a biliary tract with a pressure sensor attached thereto suggested by the present disclosure is used, the patient may consistently identify the state of the biliary tract. By doing this, the patient does not need to regularly visit the hospital so that it is possible to relieve the patient's inconvenience and outpatient medical expenses.

The effects of the present disclosure are not limited to the technical effects mentioned above, and other effects which are not mentioned can be clearly understood by those skilled in the art from the following description

### BRIEF DESCRIPTION OF THE DRAWINGS

The above and other aspects, features and other advantages of the present disclosure will be more clearly understood from the following detailed description taken in conjunction with the accompanying drawings, in which:

FIG. 1 is a view for explaining an atresia of bile ducts;

FIG. 2 is a flowchart of a method for monitoring biliary obstruction using a stent for a biliary tract with a pressure sensor attached thereto suggested by the present disclosure;

FIGS. 3 and 4 are views for explaining a stent for a biliary tract and a subcutaneous implant medical device according to an exemplary embodiment of the present disclosure;

FIG. 5 is a view for explaining a power transfer process between a subcutaneous implant medical device according to an exemplary embodiment of the present disclosure and a pressure sensor of a stent for a biliary tract; and

FIG. 6 is a view for explaining a user screen of a device which monitors biliary obstruction according to an exemplary embodiment of the present disclosure.

### DETAILED DESCRIPTION OF THE PREFERRED EMBODIMENT

Those skilled in the art may make various modifications to the present disclosure and the present disclosure may have various embodiments thereof, and thus specific embodiments will be illustrated in the drawings and described in detail in the detailed description. However, this does not limit the present disclosure within specific exemplary embodiments, and it should be understood that the present disclosure covers all the modifications, equivalents and replacements within the spirit and technical scope of the present disclosure. In the description of respective drawings, similar reference numerals designate similar elements.

Terms such as first, second, A, or B maybe used to describe various components but the components are not limited by the above terms. The above terms are used only to discriminate one component from the other component. For example, without departing from the scope of the present disclosure, a first component may be referred to as a second component, and similarly, a second component may be referred to as a first component. A term of and/or includes a combination of a plurality of related elements or any one of the plurality of related elements.

It should be understood that, when it is described that an element is “coupled” or “connected” to another element, the element maybe directly coupled or directly connected to the other element or coupled or connected to the other element through a third element. In contrast, when it is described that an element is “directly coupled” or “directly connected” to another element, it should be understood that no element is present therebetween.

Terms used in the present application are used only to describe a specific exemplary embodiment, but are not intended to limit the present disclosure. A singular form may include a plural form if there is no clearly opposite meaning in the context. In the present disclosure, it should be understood that terminology “include” or “have” indicates that a feature, a number, a step, an operation, a component, a part or the combination thoseof described in the specification is present, but does not exclude a possibility of presence or addition of one or more other features, numbers, steps, operations, components, parts or combinations, in advance.

If it is not contrarily defined, all terms used herein including technological or scientific terms have the same meaning as those generally understood by a person with ordinary skill in the art. Terms defined in generally used dictionary shall be construed that they have meanings matching those in the context of a related art, and shall not be construed in ideal or excessively formal meanings unless they are clearly defined in the present application.

Hereinafter, exemplary embodiments according to the present disclosure will be described in detail with reference to accompanying drawings.

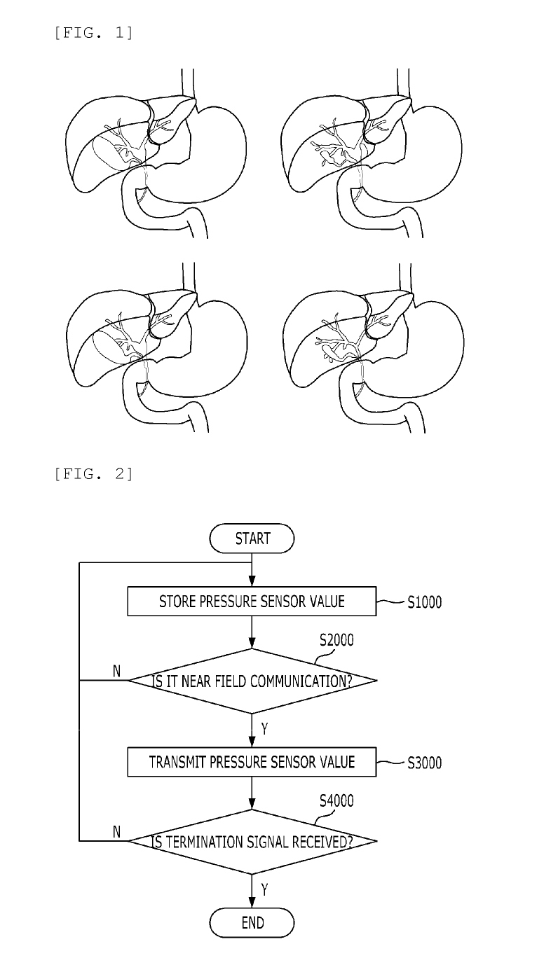

FIG. 1 is a view for explaining an atresia of bile ducts.

An atresia of bile ducts or biliary obstruction is a rare disease which occurs in the gastrointestinal tract and refers to a disease characterized by absence or destruction of a part or all of a bile duct which is a passage of bile carrying the bile from liver to the gallbladder to secrete the bile into the small intestine.

The bile is secreted from the liver and plays an important role to carry waste products from the liver and break down the fat in the small intestine. Therefore, even though the exact cause is unknown, when the bile duct is absent or obstructed, the bile is abnormally accumulated in the liver. Due to the accumulation of bile, newborns may develop jaundice of yellowing the whites of the eye and the skin and liver cirrhosis and in some cases, as additional anomalies, heart defects and kidney and spleen deformities may be accompanied.

The obstruction of the bile duct is the most common cause of chronic liver disease in newborns. Although the incidence of the obstruction of the bile duct is the same for both males and females, some studies have reported that this disease is more common for the females. In the United States, approximately one occurrence per 10000 to 15000 births is reported and approximately 400 to 600 new cases occur every year in the United States. In Europe, one occurrence per approximately 12000 births has been reported.

The obstruction of bile duct is mainly evident at 2 to 4 weeks of birth, and is accompanied by jaundice of yellowing the whites of the eyes and the skin, abnormal white stools, dark urine, sagging stomach, and hepatomegaly. At 6 to 10 weeks, additional symptoms may appear, which results in itching, instability, delayed growth, and portal hypertension that a blood pressure in the veins from the liver is increased. In some cases, the bile ducts existing in the liver may be affected. When the patient is not properly treated at this time, cirrhosis may be caused and eventually liver failure may occur.

In the case of some children patients, situs inversus that organs in the body are located on the opposite side of the normal body, levocardia that other organs are displaced, but the heart is located in the normal position (left), or congenital anomalies of the heart such as ventricular defects or congenital anomalies of the kidneys may be accompanied. As additional symptoms, an anatomical anomaly such as asplenia referring to absence of spleen or polysplenia having one or more spleens may be accompanied.

Even though the exact cause of the biliary obstruction is not known, immunological, infectious, hereditary, and environmental factors are thought as causes of the disease. Inmost cases, it is believed that the bile ducts are normal at birth, but the bile ducts are damaged due to certain causes and replaced by fibrous tissue. It has been studied that several viruses such as cytomegalovirus, reovirus type 3, and rotavirus are causative factors of this disease. The atresia of bile ducts is not a hereditary disease, but rarely several patients occur in one family.

The diagnosis of atresia of bile ducts is confirmed by laparotomy, cholangiography, and microscopic examination of a liver tissue. During the operation, a contrast agent is injected into a common bile duct and then the movement of the contrast agent which is delivered through the liver, the bile duct, and the small intestine is identified while watching the bile duct through an X-ray photograph. By doing this, the structure of the bile duct can be examined and which part is obstructed may be determined. Further, through the blood tests, the elevated levels of liver enzymes and bilirubin and the viruses or bacteria may be detected.

In order to remove the obstructed part of the bile duct and to flow the bile into the bile duct and the small intestine, Kasai's operation may be attempted to excise the occluded biliary tract outside the liver and connect the small intestine to the portal (entrance). The exact operation process may vary depending on the property and the location of the occluded bile duct. In most cases, the bile may be secreted through the operation, but some patients experience some degree of liver dysfunction after a successful operation.

The Kasai's operation is used as an initial intervention to help the growth of the child, but in spite of the Kasai's operation, liver transplantation may be ultimately needed in most cases. Further, postoperative complications may lead to cholangitis. Patients who have progressed to liver cirrhosis after Kasai's operation or who have already progressed to the liver cirrhosis at the time of diagnosis require the liver transplantation operation and the disease may be completely cured through the operation.

In addition to the non-conservative operations, there is an operation using a stent for a biliary tract as conservative operations to ensure the openness of the stenosed biliary tract. The stent for a biliary tract is manufactured to be inserted into a stenosed region of the biliary tract through an insertion tool such as a catheter and expands the stenosed region by the elastic property of the stent in the stenosed region.

More specifically, the stent refers to a mesh type structure which is used to smooth the flow of the blood vessels or tracts contracted due to the stenosis. The implantation of the stent may alleviate the symptoms due to the stenosis, but may cause an immune response so that an outer surface of the stent maybe encapsulated. However, the space secured by the stent may be obscured again due to the encapsulation and the restenosis has become major problems in most stent-related procedures.

The stent for a biliary tract is used to be inserted into an obstructed biliary tract due to various malignant diseases such as cancer of papilla, gallbladder cancer, pancreatic cancer, malignant lymph node metastasis around the bile duct, metastatic carcinoma to alleviate the obstruction. In the case of the stent for a biliary tract, restenosis may occur again on average within 4 to 5 months due to the formation of a bacterial substrate known as sludge.

However, the stent for a biliary tract of the related art is simply used only for the purpose of expanding the obstructed biliary tract. This treatment method is inconvenient because even though the biliary tract is obstructed again due to the closed stent, only when symptoms such as jaundice and fever occur, the obstruction may be diagnosed, so that the test needs to be regularly performed for early diagnosis.

Further, even though the patient regularly visits the hospital to take the examination, when the biliary tract is obstructed in the middle of the visiting cycle, severe complications such as liver damage, cholangitis, or sepsis are caused to seriously affect the life. In order to prevent this problem, the patient needs to frequently visit the hospital, which is very inconvenient.

In order to solve the above-described problem, the present disclosure suggests a stent for a biliary tract obtained by attaching a pressure sensor to the existing stent for a biliary tract. When the stent for biliary stent suggested by the present disclosure is used, the patient may conveniently diagnose the pressure in the biliary tract by him/herself without regularly visiting the hospital.

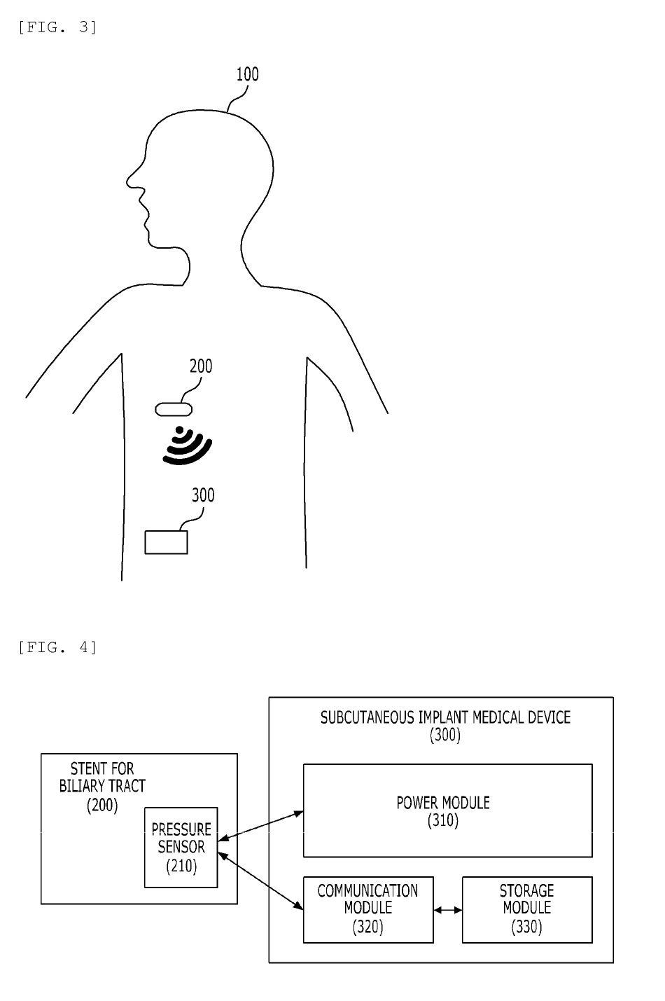

FIG. 2 is a flowchart of a method for monitoring biliary obstruction using a stent for a biliary tract with a pressure sensor attached thereto suggested by the present disclosure.

Referring to FIG. 2, the method for monitoring biliary obstruction suggested by the present disclosure involves the steps after implanting a stent for a biliary tract with a pressure sensor mounted therein into the biliary tract. First, a pressure in the biliary tract is measured to store a pressure sensor value in step S1100. To this end, an ultra-slim pressure sensor is coupled to the stent for a biliary tract.

Due to the size limitation of the stent for a biliary tract, a small-sized power device needs to be used and thus it is difficult to directly transmit the pressure sensor value to the outside from the stent for a biliary tract. In order to solve this problem, according to the present disclosure, a subcutaneous implant medical device is used to receive the pressure sensor value, in addition to the stent for a biliary tract.

Further, the power is supplied wirelessly through the subcutaneous implant medical device. The subcutaneous implant medical device accumulates and stores the pressure sensor values at a predetermined cycle and then transmits the pressure sensor values to a separate external device. For example, the subcutaneous implant medical device transmits the pressure sensor value through wireless communication with a smart phone of a patient.

During this process, the stent for a biliary tract transmits the pressure sensor value in step 51300 only when the communication with the subcutaneous implant medical device is available in step S1200. This process may be repeatedly performed until there is a termination signal in step S1400. The pressure in the biliary tract may be periodically checked through the processes.

When the subcutaneous implant medical device transmits the pressure sensor value through wireless communication with an extracorporeal device, for example, a smart phone of the patient, an application of the smart phone may monitor whether the biliary tract is re-obstructed using the pressure sensor value. Further, it is possible to suggest a timing to visit the hospital to the user while monitoring a progress of a pressure change in the biliary tract based on the pressure sensor value.

By doing this, even though the patient does not frequently visit the hospital, the re-obstruction of the biliary tract may be prevented in advance. Further, when the patient visits the hospital, the patient may help the diagnosis by providing monitoring information to the doctor. It may help the health of the patient.

That is, the present disclosure is configured by a primary communication and power transfer process between the stent for a biliary tract with a pressure sensor mounted therein and the subcutaneous implant medical device and a secondary communication and power transfer process between the subcutaneous implant medical device and the external device (for example, a server or a smart phone) separately from the primary communication and power transfer process.

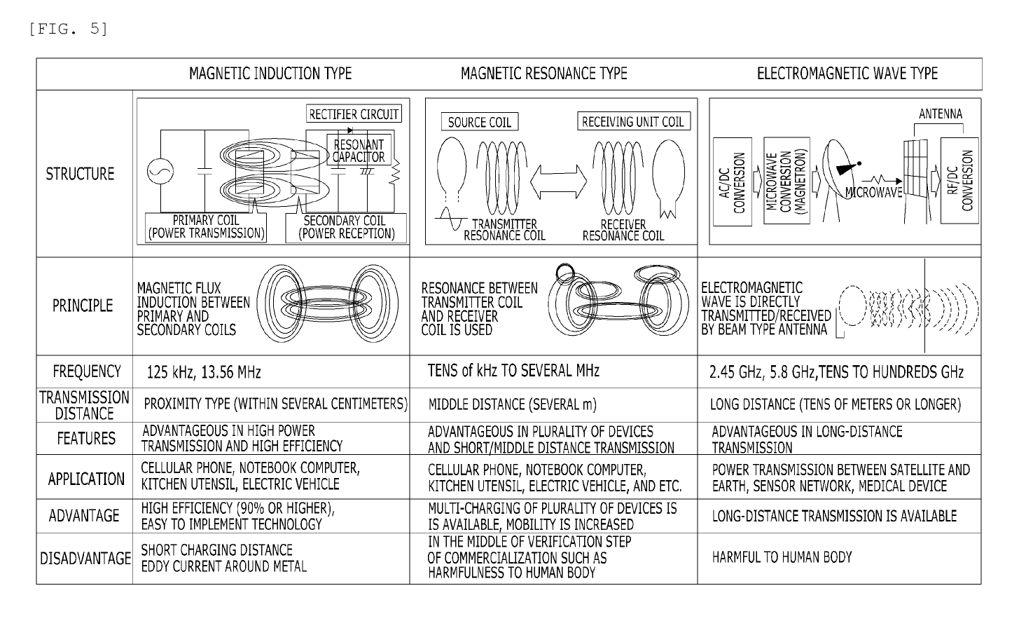

FIGS. 3 and 4 are views for explaining a stent for a biliary tract and a subcutaneous implant medical device according to an exemplary embodiment of the present disclosure.

Referring to FIG. 3, it can be seen that two devices are implanted inside the patient 100. One is a stent 200 for a biliary tract. The stent with a pressure sensor mounted therein is inserted into the obstructed biliary tract, a pressure in the biliary tract is measured periodically or in accordance with a control signal, and the pressure is transmitted to a subcutaneous implant medical device 300.

The subcutaneous implant medical device 300 serves to receive a pressure sensor value from the stent 200 for a biliary tract and to supply a power to the stent for a biliary tract 200 wirelessly. By doing this, it is possible to prevent a situation that the operation is performed to replace a power source of the pressure sensor even though the biliary tract is not re-obstructed.

In some cases, the subcutaneous implant medical device 300 may produce the power by itself so that a situation that a separate operation is performed to supply a power to the subcutaneous implant medical device 300 may be avoided. An insertion position of the subcutaneous implant medical device 300 is not specifically limited, but aside portion of the patient 100 is desirable in consideration of the distance from the stent 200 for a biliary tract.

Referring to FIG. 4, the stent 200 for a biliary tract includes a pressure sensor 210. In this case, a battery is necessary to drive the pressure sensor 210 and a human body implantable battery may be used. The human body implantable battery is not inserted into the intestine or the liver, but is inserted into the subcutaneous implant medical device 300 to supply a power source to the pressure sensor 210 of the stent 200 for a biliary tract.

In this case, as a method for transferring the power, an induced current may be used. That is, the pressure sensor 210 includes a coil. When a power module 310 of the subcutaneous implant medical device 300 generates a magnetic field, an induced current flows through the coil of the pressure sensor 210 and thus an induced electromotive force may be generated.

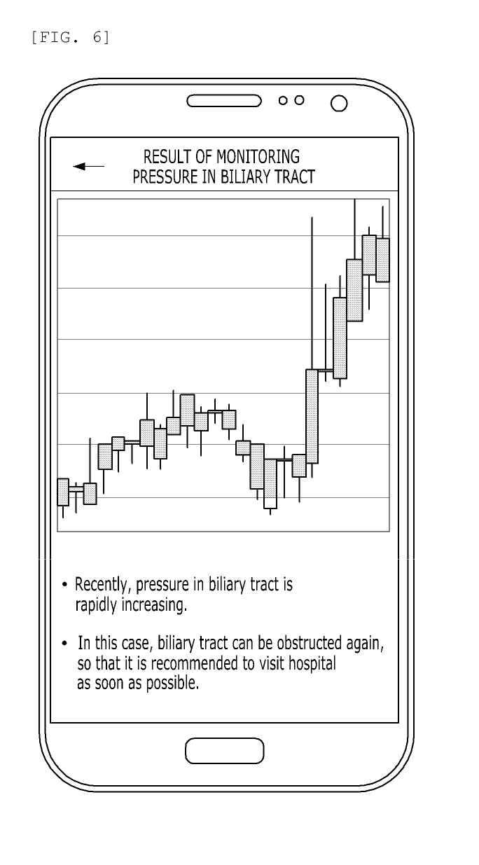

In this case, the induced current is generated to attenuate a change of the magnetic field generated in the power module 310 of the subcutaneous implant medical device 300. For example, an ultra-small magnet is disposed in the power module 310. When the ultra-small magnet moves in accordance with the movement of the patient 100, a change in the magnetic field applied to the coil in the pressure sensor 210 causes the induced current. The power transfer process between the pressure sensor of the stent for a biliary tract and the subcutaneous implant medical device 300 will be described in more detail with reference to FIG. 5.

Further, the power module 310 of the subcutaneous implant medical device 300 supplies the power through eco-friendly energy harvesting and the power maybe transferred to the pressure sensor 210 through wireless supply. For example, the power module 310 may include a thin film type solar cell. By doing this, even though the battery of the subcutaneous implant medical device 300 is discharged, a situation that the additional operation is performed to replace the battery may be avoided.

When the subcutaneous implant medical device 300 is implanted in the skin to be shallow, a part of light passes through the skin to transfer the energy to the solar cell included in the power module 310 of the subcutaneous implant medical device 300. Although it is still a research stage, it has been proved that a solar cell of 0.07 cm² is inserted into a laboratory mouse to produce a power of approximately 0.6 mW.

Further, referring to FIG. 4, the subcutaneous implant medical device 300 includes a communication module 320 as well as the power module 310. The communication module 320 receives a pressure sensor value through the wireless communication with the pressure sensor 210 and transmits the collected pressure sensor value through wireless communication with a separate external device outside the body. During this process, statistical information is generated through simple calculation and the amount of data to be transmitted may be reduced.

Wireless communication such as near field communication (NFC) or Bluetooth may be available through the communication module 320. By doing this, a value of the pressure sensor may be provided by communicating with a separate external device at a timing when the patient 100 wants to know.

During this process, the subcutaneous implant medical device 300 may further include a storage module 330 for storing pressure measurement values received from the pressure sensor 210. Pressure values which have not been transmitted to an external device are stored in the storage module 330 and then the pressure values are stored through the communication with the external devices later and corresponding data is removed to secure a storage space.

Further, the storage module 330 may further calculate statistics of the above-described pressure values, for example, information such as a minimum value, a maximum value, an average of daily basis pressure values to store the information. Such statistic information may be further transmitted at the time of communication with the device outside the body.

That is, in the related art, the patient 100 who underwent a procedure of the stent for a biliary tract due to the biliary obstruction needs to regularly visit the hospital to prevent reoccurrence of the biliary obstruction symptom. However, when the method suggested by the present disclosure is used, the patient 100 may prevent the biliary obstruction through self-diagnosis without visiting the hospital.

That is, in the case of reoccurrence of the biliary obstruction symptom, a prompt treatment can be performed before occurrence of severe complications, as compared with the treatment using a stent of the related art. By doing this, the lifespan of the patient may extend and medical expense related to the biliary obstruction may be reduced.

FIG. 5 is a view for explaining a power transfer process between a subcutaneous implant medical device according to an exemplary embodiment of the present disclosure and a pressure sensor of a stent for a biliary tract.

Referring to FIG. 5, three methods for wirelessly transferring a power are exemplified. A first method is a magnetic induction type, a second method is a magnetic resonance type, and a third method is an electromagnetic wave type. Due to harmfulness problem for the human body and a transfer distance problem, it is difficult to use the electromagnetic wave for an implant device and practically, the magnetic induction type and the magnetic resonance type may be applied to the present disclosure.

The magnetic induction type is an electromagnetic induction technique which is a contactless technique widely used for many portable terminals. When two coils are disposed to be adjacent to each other and then current flows through one coil, a magnetic flux generated at this time generates electromagnetic force from the other coil. According to the induction type, the transfer distance is within several mm and an induced current is generated in two coils which are not connected to each other by a wire, but are adjacent to each other such that a power supply source and the device are very close to each other, to charge the battery.

The magnetic induction type is a currently used technique and has an excellent energy transfer efficiency. However, the magnetic induction between two coils is sensitive to the distance between coils and the relative position so that there is a disadvantage in that when the distance between two coils is slightly increased or shifted, the transfer efficiency is rapidly deteriorated. Therefore, as a method available only for a proximate distance, an application device having transmission/reception distance which is approximately 10 mm or several mm may be found.

It was first announced in the world that the magnetic resonance type can be used as a power supply technique in November 2006 by a research team led by Marin Soljacic, assistant professor of physics at MIT, US. A couple-mode theory which is a basic concept of the magnetic resonance technique has been already introduced. A transmission/reception coil having a diameter of 60 cm is used to light up a lamp which is 2 m away from the coil so that “wireless power transmission technology” has received intensive attention again in the world and many papers have been published at the societies. Further, efforts are being made to standardize the wireless energy transmission technique and revitalize the industry therefor centered on related industries and related organizations.

Even though this technology which uses a resonance phenomenon has no effect on a long distance, when a transmission device is installed on a ceiling in a typical house, the lights may be turned on without using wires in the house, a notebook computer or a game player may be used without having a battery, and a cellular phone or an MP3 player is automatically charged so that it can be efficiently used to automatically charge household information appliances. The disadvantages of this technology are that a size of the transceiver is 60 cm, and the energy transfer efficiency is just 40 to 45%. Therefore, further researches are necessary and it is expected that miniaturization and high energy efficiency are necessary for commercialization. Further, as an object to be achieved, a necessary frequency needs to be secured.

As specific examples in which the above-described technique is implemented, there is a near field communication-wireless identification and sensing platform (NFC-WISP). The near field communication (NFC) is an international standard which enables financial payment and RFID service through short distance communication within 10 cm using a separate NFC chip and refers to a combination of a smart card and a reader. NFC is one of wireless recognition techniques extending ISO/IEC 14443 contactless card or RFID standard.

Specifically, the NFC advantageously extends ISO/IEEE 14443 standard to communicate with not only the NFC device, but also existing ISO/IEEE 14443 reader or a smart card. Recently, a technical interest for the NFC is increased in accordance with rapid spread of mobile devices such as smart phones or smart pads. Specifically, RFID 2.0 technique is increasingly utilized in the telecommunication industries and financial industries because the data can be transferred between devices without using a communication network.

Prior to the NFC, there were short range wireless communication technologies such as Bluetooth or ZigBee. However, due to the lack of the infrastructure for implementing this technology and terminals which can provide the near field communication, the utilization rate of general users is low. However, recently, the NFC technology is attracting attention due to factors such as expansion of related terminals such as mobile devices, development of technology, and various markets.

When an antenna for the NFC and an antenna for wireless charging are designed as one antenna, a communication module of a pressure sensor and a module for receiving power may be made smaller and lighter. With regard to this, Samsung Electronics and LG Electronics are filing Patent Applications. For example, there is invention of “wireless antenna for wireless charging and nfc communication and wireless terminal to which same is applied” (WO2017007231 A1).

FIG. 6 is a view for explaining a user screen of a device which monitors biliary obstruction according to an exemplary embodiment of the present disclosure.

Referring to FIG. 6, a screen which provides a monitoring result obtained by analyzing data may be seen from a smart phone which receives a pressure sensor value collected from the subcutaneous implant medical device 300. The values of the pressure sensor are illustrated in the form of a candle chart for every day and thus a progress of a pressure change in the biliary tract may be grasped at a glance.

Further, information related to the analysis result maybe provided to the patient 100 as illustrated in a lower portion of FIG. 6. At the lower portion of FIG. 6, a guidance message such as “Recently, pressure in biliary tract is rapidly increasing” and “In this case, biliary tract can be obstructed again, so that it is recommended to visit the hospital as soon as possible” is exposed.

As described above, the progress of the pressures in the biliary tract is provided through a separate external device which is possessed by the patient 100 so that the pressure change in the biliary tract is easily self-diagnosed and thus the re-obstruction of the biliary tract may be prevented in advance.

In order to provide analysis information, the external device such as a smart phone may manage the pressure values in the biliary tract by dividing the pressure values into sections in advance. For example, the range of the pressure values is divided into a normal level, a warning level, and a risk level to be compared with the value received from the subcutaneous implant medical device 300. By doing this, the guidance information may be provided to the user.

The exemplary embodiments of the present disclosure have been described with reference to the accompanying drawings, but those skilled in the art will understand that the present disclosure may be implemented in another specific form without changing the technical spirit or an essential feature thereof. Thus, it is to be appreciated that the embodiments described above are intended to be illustrative in every sense, and not restrictive.

What is claimed is:

1. A system for monitoring a pressure in a biliary tract, comprising:
a stent for a biliary tract including a pressure sensor; and
a subcutaneous implant medical device including a communication module which receives a measured value of a pressure in a biliary tract through communication with the pressure sensor and a power module which supplies a power to the pressure sensor.

2. The system for monitoring a pressure in a biliary tract according to claim 1, wherein the communication module communicates with the pressure sensor using any one of near field communication (NFC) and Bluetooth.

3. The system for monitoring a pressure in a biliary tract according to claim 1, wherein the pressure sensor includes a coil which generates an induced current and the power module wirelessly supplies the power to the coil through electromagnetic induction.

4. The system for monitoring a pressure in a biliary tract according to claim. 1, wherein the power module includes a thin film for harvesting solar energy and supplies the solar energy to the pressure sensor.

5. The system for monitoring a pressure in a biliary tract according to claim 1, wherein the subcutaneous implant medical device further includes a storage module and
the measured value of the pressure and statistic information of the measured value of the pressure are stored in the storage module.

6. The system for monitoring a pressure in a biliary tract according to claim 1, wherein the subcutaneous implant medical device is inserted under a subcutaneous tissue of a lateral abdominal region.

7. The system for monitoring a pressure in a biliary tract according to claim 1, further comprising:
an external device which receives the measured value of the pressure from the communication module, analyzes the received measured value of the pressure to expect a timing of biliary obstruction, and provides expected information to a patient.

8. A method for monitoring a pressure in a biliary tract, comprising:
receiving a measured value of a pressure in a biliary tract from a stent for a biliary tract having a pressure sensor mounted therein, by a subcutaneous implant medical device;
collecting the measured value of the pressure at a predetermined cycle and transmitting collected information to an external device, by the subcutaneous implant medical device; and
analyzing the collected information to expect a timing of biliary obstruction, and providing expected information to the patient, by the external device.
